(19) United States
(12) Patent Application Publication
(10) Pub. No.: US 2002/0137731 A1
(43) Pub. Date: Sep. 26, 2002

(54) Combination of radiation and vitamin D3 analogs for the treatment of cancer

(76) Inventor: David A. Gewirtz, Richmond, VA (US)

Correspondence Address: FULBRIGHT & JAWORSKI L.L.P., A REGISTERED LIMITED LIABILITY PARTNERSHIP, 600 CONGRESS AVENUE, SUITE 2400, AUSTIN, TX, 78701, US

(21) Appl. No.: 09/870,930

(22) Filed: May 30, 2001

Related U.S. Application Data
(60) Provisional application No. 60/207,290, filed on May 30, 2000.

(51) Int. Cl. A61K 31/59; A61N 5/00

(52) U.S. Cl. 514/167; 600/1

(57) ABSTRACT

The present invention is drawn to the use of a vitamin D₃ analogs in combination with radiation therapy for the treatment of hyperproliferative diseases, including cancer. Such combination therapies may be combined with additional therapies such as chemotherapy, immunotherapy, gene therapy or surgery.

### BACKGROUND OF THE INVENTION

The present application claims priority to U.S. Ser. No. 60/207,290, filed May 30, 2000, the entire text of which is specifically incorporated by reference herein without disclaimer.

1. Field of the Invention

The present invention relates generally to the fields of cancer biology and radiotherapy. More particularly, it concerns the use of vitamin D₃ analogs, in combination with radiation, to treat various forms of cancer.

2. Description of Related Art

Cancer is one of the most devastating health problems in the world today, affecting 1 in 5 individuals in the United States. Thus, curbing the growth of neoplastic cells is a major focus of the medical research community. Research has led to the discovery of novel therapies, including cytotoxic agents commonly employed in chemotherapy include anti-metabolic agents interfering with microtubule formation, alkylating agents, platinum-based agents, anthracyclines, antibiotic agents, topoisomerase inhibitors, and others. In addition, the more traditional surgical and radiation therapies have been refined, while cutting edge treatments involving immune modulation and gene therapy have been developed.

While many new chemotherapeutic agents have been developed, some well known compounds have found new uses as cancer therapeutics. For example, cholecalciferol (vitamin D) has been shown to effect differentiation and reduce proliferation of several cell types cells both in vitro and in vivo. The active metabolite of vitamin D (1,25-dihydroxycholecalciferol (hereinafter “1,25D₃ ”) and analogs (e.g., 1,25-dihydroxy-16-ene-23-yne-cholecalciferol; 1,25-dihydroxy-16-ene-23-yne-26,27-hexafluoro-19-nor-cholecalciferol) mediate significant in vitro and in vivo anti-tumor activity by retarding the growth of established tumors and preventing tumor induction (Colston et al. (1989); Belleli et al. (1992); McElwain et al. (1995); Clark et al. (1992); Zhou et al. (1989); U.S. Pat. No. 5,145,846). In addition to retarding neoplastic growth, 1,25D₃ induces a G₀G₁-S phase block in the cell cycle (Godyn et al. (1994); Rigby et al. (1985); Elstner et al. (1995); Wang et al. (1996)). These properties have led to the successful use of 1,25D₃ to treat neoplastic tumors (Cunningham et al. (1991); Mackie et al. (1993); Bower et al. (1991)).

In addition to its antineoplastic and cell-cycle blocking effects, 1,25D₃ treatment can lead to hypercalcemia. As a result, 1,25D₃ is typically administered for therapeutic applications (e.g., metabolic bone disease) at relatively low doses (e.g., about 1 μg/day to about 2 μg/day) long term. To mitigate the effects of hypercalcemia, analogs have been developed which retain antiproliferative activity without inducing hypercalcemia (e.g., Zhou et al (1991); Binderup et al. (1991); Binderup et al. (1991)). Many of these synthetic analogs are more potent than 1,25D₃ in inhibiting neoplastic growth (for a review of many such analogs, see Calverley et al. (1992)).

There have been attempts to develop combination drug protocols based, in part, on vitamin D analogs. For example, the inhibitory effect of concurrent combination of 1,25D₃ and platinum drugs on the growth of neoplastic cells has been studied (Saunders et al. (1993); Cho et al. (1991)), and similar work has focused on concurrent combinations of 1,25D₃ and other cytotoxic agents (Tanaka et al. (1989)). The application of these approaches in therapy would require the long-term application of high doses of 1,25D₃ in some protocols, which, as mentioned, can precipitate significant side effects. Other studies have shown that combination with specific cytotoxic agents (e.g., paclitaxel and cyclophosphamide) are effective (U.S. Pat. No. 6,087,350).

There remains a clear need to for improved cancer therapies. However, as demonstrated above, it is not possible to predict which cancer therapies can be effectively combined with vitamin D₃ in the treatment of cancer, and which vitamin D₃ analogs will be effective in such combinations.

### SUMMARY OF THE INVENTION

Thus, in accordance with the present invention, there is provided a method of inducing cell death in a cancer cell comprising administering to said cell:

a vitamin D₃ compound having the structure:

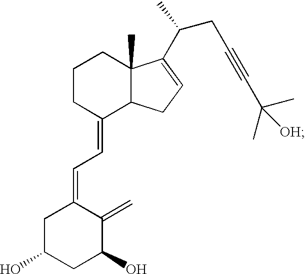

(ILEX 7553) and ionizing radiation,

both in sufficient doses that, when combined, cell death is induced.

The radiation may be x-irradiation or γ-irradiation. The vitamin D₃ compound is provided prior to said radiation, after said radiation or concurrent with said radiation. The vitamin D₃ compound may be administered less than one hour prior to said radiation, less than four hours prior to said radiation, less than eight hours prior to said radiation, less than 12 hours prior to said radiation, less than 24 hours prior to said radiation or less than 48 hours prior to said radiation. Alternatively, the vitamin D₃ compound may be administered less than one hour after said radiation, less than four hours after said radiation, less than eight hours after said radiation, less than 12 hours after said radiation, less than 24 hours after said radiation, or less than 48 hours after said radiation.

The method of claim 1, wherein said vitamin D₃ compound is formulated as about 0.5 to 1.0% of a pharmaceutical composition, or as about 0.8% of a pharmaceutical composition. The ionizing radiation may be x-irradiation provided at fractionated doses of 2 Gy, at a total dose of about 40 Gy or about 60 Gy, or at a total dose of about 40 to 60 Gy.

The cancer cell may be a brain cancer cell, a liver cancer cell, a pancreatic cancer cell, a leukemia cell, a lymphatic cancer cell, a head & neck cancer cell, a lung cancer cell, a breast cancer cell, a thyroid cancer cell, a prostate cancer cell, a stomach cancer cell, a esophageal cancer cell, a colon cancer cell, a rectal cancer cell, a testicular cancer cell, a bladder cancer cell, a cervical cancer cell, an ovarian cancer cell or a skin cancer cell. In particular, the cancer cell is selected from the group consisting of an acute promyelocytic leukemia cell, a colon cancer cell, a rectal cancer cell and a liver cancer cell.

In another embodiment, there is provided a method of treating a patient with cancer comprising administering to said patient ILEX 7553 and ionizing radiation, both in sufficient doses that, when combined, cancer cell death is induced, thereby treating cancer.

The radiation may be x-irradiation or γ-irradiation. The vitamin D₃ compound may be provided prior to said radiation, after said radiation or concurrent with said radiation. The vitamin D₃ compound may be is administered about one hour prior to said radiation up to 48 hours prior to said radiation or administered less than one hour up to 48 hours after said radiation.

The method vitamin D₃ compound may be provided at dose range of about 0.01 μg/kg body weight to about 2.5 μg/kg body weight, at dose range of about 0.1 μg/kg body weight to about 2.5 μg/kg body weight, at dose range of about 0.5 μg/kg body weight to about 2.5 μg/kg body weight, at dose range of about 1.0 μg/kg body weight to about 2.5 μg/kg body weight, or at dose range of about 0.01 μg/kg body weight to about 1.0 μg/kg body weight.

The ionizing radiation may be x-irradiation provided at fractionated doses of 2 Gy or at a total dose of about 40 to 60 Gy. The method of claim 26, wherein said cancer is a brain cancer, a liver cancer, a pancreatic cancer, a leukemia, a lymphatic cancer, a head & neck cancer, a lung cancer, a breast cancer, a thyroid cancer, a prostate cancer, a stomach cancer, a esophageal cancer, a colon cancer, a rectal cancer, a testicular cancer, a bladder cancer, a cervical cancer, an ovarian cancer and a skin cancer. In particular, the cancer is selected from the group consisting of acute promyelocytic leukemia, colon cancer, rectal cancer and liver cancer. The patient may further be treated with surgery, chemotherapy, immunotherapy, gene therapy or hormonal therapy. The patient may previously have received a cancer therapy. The cancer may be recurrent cancer.

In yet another embodiment, there is provided a method of rendering a non-resectable tumor resectable comprising administering to a patient having said tumor ILEX 7553 and ionizing radiation, both in sufficient doses that, when combined, tumor cells are killed and said tumor is rendered resectable. The method may further comprise resecting said tumor.

In still yet another embodiment, there is provided a method of treating a patient with metastatic cancer comprising administering to said patient ILEX 7553 and ionizing radiation, both in sufficient doses that, when combined, cancer cell death is induced, thereby reducing the number of metastases.

In still an even further embodiment, there is provided a method of reducing the tumor burden in a patient comprising administering to said patient ILEX 7553 and ionizing radiation, both in sufficient doses that, when combined, tumor cells are killed and said tumor burden is reduced.

In yet an additional embodiment, there is provided a method of sensitizing a tumor cell to radiation comprising (a) selecting a tumor cell for radiation therapy and (b) administering to said tumor cell ILEX 7553. Similarly, there is provided a method of reducing the radiation dose response curve for a tumor cell comprising (a) selecting a tumor cell for radiation therapy and (b) administering to said tumor cell ILEX 7553. Also provided is a method of overcoming radiation resistance in a tumor cell comprising (a) selecting a radiation resistant tumor cell for radiation therapy and (b) administering to said tumor cell ILEX 7553.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Influence of ILX 23-7553 Pretreatment Followed by Adriamycin or Ionizing Radiation on Viable Cell Number in MCF-7 Cells. Cells were treated with 200 nM ILX 23-7553 for 72 h followed by exposure to 1 μM adriamycin (acute exposure) or 10 Gy ionizing radiation. Viable cell number was determined by trypan blue exclusion 72 h after exposure to adriamycin or irradiation. Cell number was decreased by 60, 74 and 75%, respectively, after treatment with ILX 23-7553, adriamycin or irradiation alone. ILX 23-7553 pretreatment followed by adriamycin or ionizing radiation decreased viable cell number by 97 and 93%, respectively.

FIG. 2—Clonogenic Survival of MCF-7 Cells Pretreated with ILX 23-7553 Followed by Various Doses of Adriamycin (Top Panel) or Ionizing Radiation (Bottom Panel). Cells were treated with 200 nM ILX 23-7553 for 72 h followed by exposure to 1-100 nM adriamycin or 0.5-5.0 Gy irradiation. Colony number was determined 7-10 d after adriamycin or irradiation treatment. ILX 23-7553 pretreatment produced 2- and 4-fold decreases in the doses of adriamycin and irradiation required to reduce clonogenic survival by 50%.

FIG. 3—Percent TUNEL-Positive Cells. DNA fragmentation indicative of apoptosis was evident in 14 and 7% of cells, respectively, pretreated with ILX 23-7553 followed by adriamycin or irradiation. Percent TUNEL positive cells is the fraction of fluorescent cells.

### DESCRIPTION OF ILLUSTRATIVE EMBODIMENTS

Cancer remains one of the most prolific killers in industrialized countries. While surgery, radiation and chemotherapy are effective in the treatment of some cancers, many others are resistant to such therapies. Thus, there is a constant need to develop new and improved cancer therapies.

Vitamin D₃ and its analogs have shown promise in the treatment of various forms of cancer. However, these compounds have not ben shown by themselves to achieve cell killing, or “apoptosis.” They also have been used in combination with certain chemotherapeutics with mixed results. Thus, the full potential of this class of drugs as chemotherapeutics remains to be realized.

I. The Present Invention

Ionizing radiation generally fails to promote primary apoptotic response in experimental breast tumor cell lines. Similarly, the response of tumor cells to treatment with vitamin D3 and analogs thereof is growth inhibition. The present inventors have chosen to combine these two different therapies in the hope of increasing the benefit achieved by each.

As discussed in the examples, pretreatment of cancer cells with a vitamin D₃ analog ILX 23-7553, followed by radiation, decreased viable cell numbers by 66% as compared to ILX 23-7553 alone, or 24% as compared to radiation alone. It also shifted the dose response curve for clonogenic survival by 4-fold. Surprisingly, the combination therapy was found to induce DNA fragmentation and morphological changes indicative of apoptotic cell death.

II. Vitamin D₃, Analogs

Vitamin D₃ is cholecalciferol, one of the vitamin D family. Vitamin D₃ is necessary for the utilization of calcium and phosphorus, and for the assimilation of vitamin A. It also has a strong immune enhancing effect. Reports indicate that as much as half the American population is vitamin D deficient. Vitamin D is produced in the skin by exposure to sunlight and is added to milk or other dairy products. One Massachusetts hospital discovered that 57% of their patients were deficient, and 22% were severely deficient. In another study of younger people averaging 44 years of age, 42% were found to be deficient and 11% severely deficient. In the tropics, where sunshine makes vitamin D deficiency rare, osteoporosis, cataracts, colon and prostate cancer are far less common. Thus, vitamin D₃ has been recommended for use in treating bacterial infections, metabolic bone disease and certain cancers.

Vitamin D₃ appears to be more closely related to a hormone than a vitamin because of the many cellular functions it performs. Among its actions is the regulation of cellular proliferation and differentiation. Vitamin D₃ works synergistically with vitamin A to control cancer by inducing certain cancer cells to differentiate into normal cells and to stop multiplying uncontrollably. This effect is so pronounced that vitamin D₃ analogs are being developed for cancer therapy. Among the cancers that Vitamin D₃ has been shown to be effective against are colorectal, breast, prostate, ovarian, and several kinds of leukemia and lymphoma.

Anyone taking more than 1300 IU's per day should have periodic blood tests performed to be sure that not too much calcium is being absorbed or that kidney and liver function is not adversely affected. Underlying kidney disease is a contraindication. High doses should be taken under the supervision of a physician. Cancer patients should take 2000 to 3000 IU's per day on an empty stomach along with an essential fatty acid (e.g., flax oil). Subjects should obtain at least 15 to 30 minutes of sunshine directly on the skin at least 3 times per week. Adequate quantities of phytochemicals from vegetables and fruits, together with essential fatty acids, help prevent skin cancer, as does vitamin D₃ itself.

A variety of vitamin D₃ analogs have been disclosed. The following are U.S. Patents that disclose various forms of vitamin D₃, analogs:

U.S. Pat. Nos. 6,218,430, 6,177,586, 6,121,312, 6,100,294, 6,071,897, 6,043,386, 6,017,908, 6,017,907, 6,008,209, 5,919,986, 5,986,112, 5,872,113, 5,830,885, 5,786,348, 5,719,297, 5,710,294, 5,633,241, 5,618,805, 5,587,497, 5,561,123, 5,248,029, 5,342,975, 5,516,525, 5,508,274, 5,430,196, 5,403,832, 5,401,732, 5,389,622, 5,378,695, 5,376,651, 5,366,736, 5,342,975, 5,316,770, 5,225,579, 5,206,230, 5,202,266, 5,200,536, and 4,906,785.

Of particular relevance to the present invention is ILEX 23-7553, which has the following chemical structure:

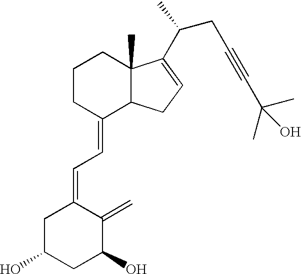

The details for this compound, including synthesis and chemical properties, may be found in U.S. Pat. No. 5,145,846, incorporated herein by reference.

III. Radiation

Radiation that causes DNA damage has been used extensively and includes what are commonly known as γ-rays, X-rays (external beam), and the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Typical dosage ranges for X-rays range from daily doses of 50, 75, 100, 150 or 200 roentgens for prolonged periods of time (3 to 4 wk), to single doses of 1000, 2000, 3000, 4000, 5000 or 6000 roentgens. Dosage ranges for radioisotopes vary widely, and depend on the half-life of the isotope and the strength and type of radiation emitted.

In accordance with the present invention, the amount of radiation may be applied in a fractionated regimen—multiple doses adding to a total doses of about 40 to 60 Gy. More particularly, the regimen may comprises fractionated individual doses of 2 Gy (200 rads). In a specific embodiment, x-irradiation is employed.

The terms “contacted” and “exposed,” when applied to a cell, are used herein to describe the process by which a radiotherapy is delivered to a target cell or placed in direct juxtaposition with the target cell. To achieve cell killing or apoptosis, the radiation is delivered to a cell in combination with a vitamin D₃ compound in an amount effective to kill the cell or induce apoptosis.

The two main types of radiation include external beam radiation and delivery of radioactive isotope internally. With regard to the latter, it is not uncommon to use a targeting agent, such as a monoclonal antibody, that carries the radionuclide to the hyperproliferative tissue. Suitable radioactive isotopes include astatine²¹¹, ¹⁴carbon, ⁵¹chromium, ³⁶chlorine , ⁵⁷cobalt, ⁵⁸cobalt, Copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰.

IV. Vitamin D₃ Analogs in Combination with Radiation

In accordance with the present invention, in order to create a more effective cancer therapy, the inventor proposes to administer vitamin D₃ analogs in combination with radiotherapy for the treatment of hyperproliferative disease, such as cancer. In particular, the therapy is designed to induce apoptosis (cell death) in cancer cells, although reducing the incidence or number of metastases, and reducing tumor size also are contemplated. Tumor cell resistance to radiotherapy agents represents a major problem in clinical oncology. Thus, in the context of the present invention, it also is contemplated that vitamin D₃ analog therapy could be used on radiation resistant lines to improve the efficacy of the latter.

Therefore, the present inventor proposes the treatment of various hyperproliferative diseases, including cancers. Specifically, cancers of the liver, pancreas, blood (leukemias), lymphatic system, brain, head & neck, lung, breast, thyroid, prostate, stomach, esophagus, colon, rectum, testes, bladder, cervix, ovaries and skin are suitable for treatment according to the present invention. In particular, renal cancer, neuroblastoma, retinoblastoma, chronic myelogenous leukemia, acute leukemia, acute promyelocytic leukemia, and non-small cell lung cancer are included.

This process may involve contacting the hyperproliferative cells with the radiation or vitamin D₃ analog at the same time. Alternatively, the vitamin D₃ analog therapy may precede or follow the radiation by intervals ranging from minutes to weeks. In embodiments where the radiation and vitamin D₃ analog are applied at distinct times, one would generally ensure that a significant period of time did not expire between the time of each delivery, such that the vitamin D₃ analog and radiation would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, vitamin D₃ analog therapy is “A” and the radiation is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the vitamin D₃ analogs of the present invention to a patient will follow general protocols for the administration of chemotherapeutics, taking into account the toxicity, if any. It is expected that the treatment cycles would be repeated as necessary.

V. Other Cancer Therapies

In accordance with the present invention, it also is envisioned that a therapy based on a combination of vitamin D₃ analog and radiation may farther be combined with other cancer therapies. Such therapies include classic chemotherapy, surgery, immunotherapy, gene therapy, and hormonal therapy. These and other therapies are described below.

a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristin, vinblastin and methotrexate, or any analog or derivative of the foregoing.

b. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. Finally, the effector may be a particular compound that interacts with or stimulates the immune system.

Immunotherapy, thus, could be used as part of a combined therapy, in conjunction with vitamin D3 analog/radiation therapy. In certain embodiments relying on immune targeting functions, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

In non-targeting embodiments, the immunomodulatory agents include agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor, interferon α, β, and γ, IL-2 and other cytokines, F42K and other cytokine analogs, or MIP-1, MIP-1beta, MCP-1, RANTES, and other chemokines.

c. Genes

In yet another embodiment, the additional treatment is a gene therapy in which a therapeutic polynucleotide is administered before, after, or at the same time as the vitamin D₃ analog and/or radiation. Delivery of a vector encoding either a full length therapeutic gene in conjunction with vitamin D₃ analog or radiation will have a combined anti-hyperproliferative effect on target tissues.

i. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

ii. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK}4 gene maps to 9p21, a chromosome region frequently deleted in many tumor types. Homozygous deletions and mutations of the p16^{INK4} gene are frequent in human tumor cell lines. This evidence suggests that the p16^{INK} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al, 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

iii. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, Al, Bfl-1) or counteract Bcl-2 function and promote cell death (e.g., Bax, Bak, Bik, Bim, Bid, Bad, Harakiri).

d. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

e. Other Agents

It is contemplated that other agents may be used in combination with the present invention to improve the therapeutic efficacy of treatment. For example, increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

VI. Pharmaceutical Formulations and Routes of Administration

Pharmaceutical compositions of the present invention comprise an effective amount of a vitamin D₃ analog and optional additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains a vitamin D₃ analog and optional additional agent will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The formulations of the present invention may comprise different types of carriers depending on whether they are to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally (as well as into the tumor vasculature, tumor bed, or lymphatic or vasculature system regional to the tumor), intramuscularly, intraperitoneally, subcutaneously, subconjunctival, intravesicularly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a vitamin D₃ analog of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active vitamin D3 analog. In other embodiments, the an active vitamin D3 analog may comprise between about 0.5%, 0.8%, 1%, 1.5%, 2%, to about 75% of the weight of the unit, or between about 0.5% to about 1%, for example. In other non-limiting examples, a dose may also comprise from about 0.01 μg/kg/body weight, about 0.05 μg/kg/body weight, about 0.10 μg/kg/body weight, about 0.50 μg/kg/body weight, about 1.0 μg/kg/body weight, about 1.5 μg/kg/body weight, about 2.0 μg/kg/body weight, up to about 2.5 μg/kg/body weight per administration, and any range derivable therein. In non-limiting examples of a derivable range from the numbers listed herein, a range of about 0.01 μg/kg/body weight to about 2.5 μg/kg/body weight, about 0.05 μg/kg/body weight to about 2.0 μg/kg/body weight, etc., can be administered, based on the numbers described above.

The composition also may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain embodiments the vitamin D₃ analog is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. In particular embodiments, prolonged absorption of an injectable composition can be brought about by the use in the compositions of agents delaying absorption, such as, for example, aluminum monostearate, gelatin or combinations thereof.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Materials:

The human breast tumor cell line, MCF-7 (p53 wild type and VDR +) was obtained from NCI, Frederick, Md. The vitamin D₃ analog, ILX-23-7553 was generously provided by ILEX Pharmaceuticals, San Antonio, Tex. RPMI 1640 and supplements were obtained from GIBCO Life Technologies, Gaithersburg, Md. Reagents used for the TUNEL assay (terminal transferase, reaction buffer, and Fluorescein-dUTP) were purchased from Boehringer Manheim, Ind. All other reagents used in the study were analytical grade.

Cell Culture:

All cell lines were grown from frozen stocks in basal RPMI 1640 medium supplemented with 10% fetal calf serum, 2 mM L-glutamine, penicillin/streptomycin at 37° C. under a humidified, 5% CO₂ atmosphere. All studies were conducted using approximately 10⁴ cells/cm² at day 0 with the use of time-equivalent and concentration-equivalent controls. The results shown are averages of two to three studies.

Trypan Blue Exclusion:

The response of cells treated with adriamycin (1 μM) for 2 hr and irradiation (10 Gy) was assessed 72 hr after treatment by trypan blue exclusion. The response of cells treated with ILX 23-7553 (200 nM) for 72 hr was evaluated 72 hr after removal of ILX 23-7553. To evaluate the combined effects of adriamycin or irradiation with ILX 23-7553 (200 nM), cells were treated with ILX 23-7553 for 72 hr prior to adriamycin (1 μM) or irradiation (10 Gy) and allowed to grow for an additional 72 hr. Cells were harvested using trypsin, stained with 0.4% trypan blue and trypan blue negative cells were counted under phase contrast microscopy.

Clonogenic Analysis:

MCF-7 cells were treated with ILX 23-7553 (200 nM), adriamycin or radiation alone and with ILX 23-7553 preceding various doses of adriamycin or radiation. For analysis of additivity, MCF-7 cells were treated with various doses of ILX 23-7553 (0-200 nM) in combination with a single dose of either adriamycin (20 nM) or irradiation (2.5 Gy). Cells were trypsinized immediately following adriamycin or radiation under sterile conditions and plated in triplicate in 6 well cell culture plates at approximately 1000 cells for each condition. After 7-10 days, the cells were fixed with 100% methanol, air-dried for 1-2 hr and stained with 01% crystal violet. Groups of 50 or more cells were counted as colonies.

Tunel:

The method of Gavrielli et al. (1992) was utilized as an independent assessment of apoptotic cell death in combined cytospins containing both adherent and non-adherent cells. The cells were fixed and the fragmented DNA in cells undergoing apoptosis was detected using the In Situ Cell Death detection Kit (Boehringer-Manheim). In this assay, the fragmented DNA in individual cells was end labeled using fluorescein-dUTP at strand breaks by the enzyme terminal transferase. The slides were then washed, mounted in Vectashield and photographed using a Nikon fluorescent microscope. For quantitation, fluorescent cells were scored positive for DNA fragmentation. These results are expressed as the number of fluorescent cells divided by the total number of cells in each field.

Cell Morphology:

Seventy-two hours after adriamycin or irradiation exposure, cells were washed and cytocentrifuged onto microscopic slides. The cells were stained Wright-Giesma stain and photographed under a Nikon light microscope.

Synergism vs. Additivity:

The predicted responses for the colony forming assay were determined using the following model: y=exp(B₀+B₁x₁+B₂x₂) where y is the predicted response, x₁ is either dose of irradiation (10 Gy) or adriarnycin (nM), x₂ is the dose of ILX 23-7553 (nM), B₀ is an unknown parameter associated with the intercept, B₁ is an unknown parameter associated with the slope of x₁ and B2 is an unknown parameter associated with the slope of x₂. Parameter estimates were found using a generalized least squares criterion for nonlinear models. A constant variance was assumed across the dose range of all four compounds. The Gauss-Newton iterative algorithm was used in PROC NLIN in SAS (version 6.12) to find parameter estimates. The compare the observed response at each combination point to that predicted under the hypotheses of additivity, a prediction interval was used following Gennings et al. (1997). An overall test for additivity (Radford et al., 1994) was based on testing they hypothesis that the mean response under the hypothesis of additivity is the true mean response. The estimated responses under the hypothesis of additivity were provided by the additivity model. The estimated responses for the true means were provided by the sample means at each mixture group.

### Example 2

### Results

Influence of ILX 23-7553 Pretreatment Followed by Adriamycin or Ionizing Radiation on Viable Cell Number and Clonogenic Survival.

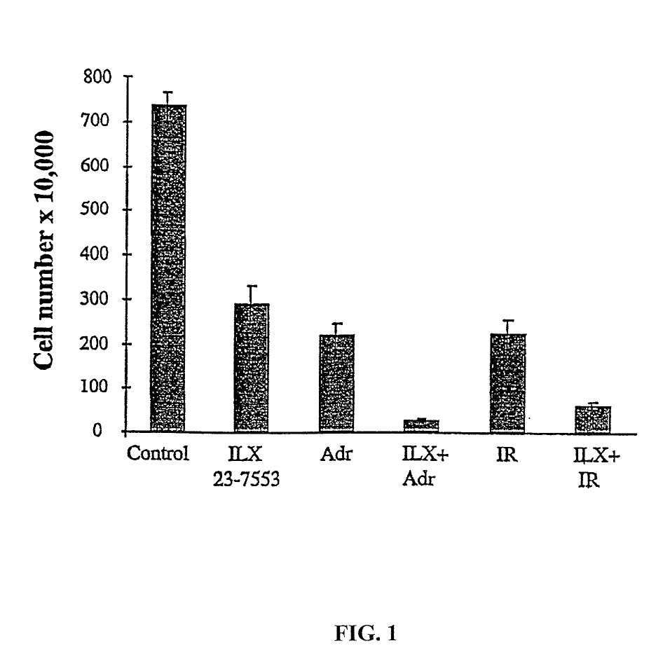

Previous studies in the laboratory have demonstrated that the Vitamin D₃ analog, EB 1089, enhances the response of MCF-7 breast tumor cells to both adriamycin and irradiation (Sundaram and Gewirtz, 1999; Sundaram et al., 2000). In order to determine if ILX 23-7553 (200 nM) pretreatment could similarly interact with adriamycin or irradiation, viable cell number was determined by trypan blue exclusion 72 hr after MCF-7 cells were treated with adriamycin (1 μM) or irradiation (10 Gy) alone or with ILX 23-7553 pretreatment. FIG. 1 indicates that viable cell number was decreased by 60%, 74% and 75%, respectively, in cells treated with ILX 23-7553, adriamycin or irradiation alone compared to growth of untreated controls. ILX 23-7553 pretreatment significantly increased the effects of both adriamycin and irradiation in reducing viable cell number. Cells treated with ILX 23-7553 in combination with adriamycin or irradiation demonstrated reductions of 97 and 93%, respectively, in viable cell number.

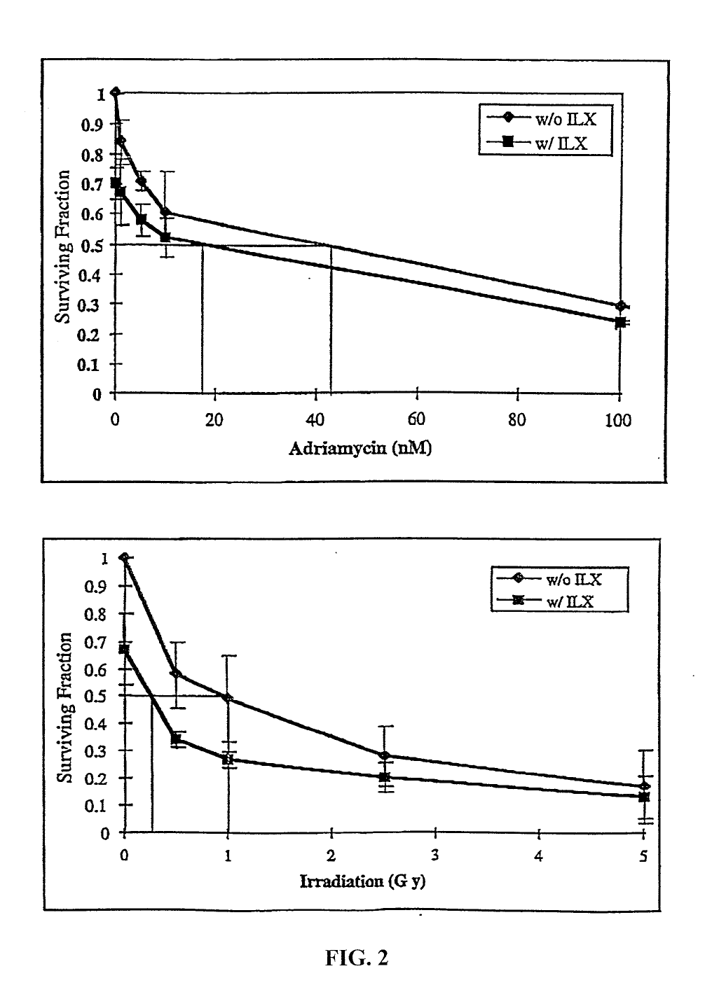

The next series of studies were performed in order to determine if these effects in MCF-7 cell growth translated into effects on clonogenic survival. MCF-7 cells were exposed to adriamycin concentrations ranging from 1 to 100 nM or 0.5 to 5 Gy ionizing radiation with and without 200 nM ILX 23-7553 pretreatment. FIG. 2A demonstrates that the concentration of adriamycin required to reduce clonogenic survival by 50% was decreased almost 2.5-fold by ILX 23-7553 pretreatment declining from 45 nM to 18 nM. The sensitivity of MCF-7 cells to ionizing radiation was more profoundly shifted by ILX 23-7553 such that there was a 4-fold reduction in the dose or irradiation required to produce a 50% decrease in clonogenic survival from 1 Gy to 0.25 (FIG. 2B). Thus, ILX 23-7553 in combination with either adriamycin or irradiation was more effective at decreasing clonogenic survival and viable cell number in MCF-7 cells than treatment with any of the agents alone.

Influence of ILX 23-7553 Pretreatment Followed by Adriamycin or Irradiation on the Incidence of Apoptotic Cell Death.

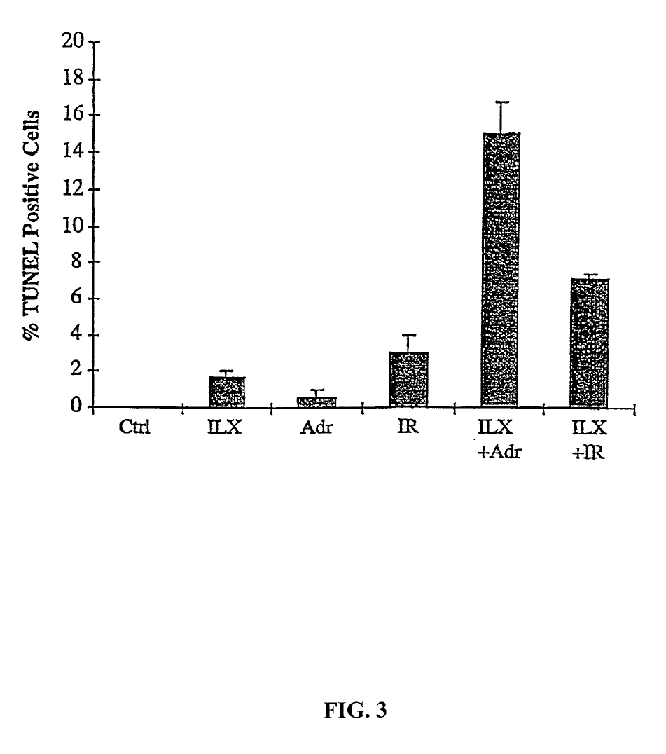

Previous work has shown that neither adriamycin nor irradiation induces apoptotic cell death in MCF-7 cells (Fomari et al., 1994; Fornari et al., 1996; Gewirtz et al., 1998; Watson et al., 1997; Whitacre and Berger, 1997; Wilson et al., 1995; Wosikowski et al., 1995). In order to determine if the enhanced ability of adriamycin and irradiation to decrease cell viability and clonogenic survival when preceded by ILX 23-7553 treatment could be attributed to an increased incidence of apoptosis, the terminal transferase end labeling (TUNEL) assay was performed. DNA fragmentation indicative of apoptotic cell death was not induced by adriamycin, irradiation or ILX 23-7553 alone. In contrast, ILX 23-7553 pretreatment followed by adriamycin or irradiation increased the number of cells with DNA fragmentation to 15% and 7% cells, respectively. FIG. 3.

In addition to DNA fragmentation, apoptosis is characterized by morphological changes including condensation of the cell cytoplasm and the formation of apoptotic bodies. An analysis of cell morphology was performed to assess whether ILX 23-7553 pretreatment followed by adriamycin or irradiation could induce such morphological changes. Apoptotic cells which appear dark and shrunken compared to normal cells were observed after exposure to ILX 23-7553 in combination with both adriamycin and irradiation. Treatment with adriamycin, irradiation or ILX 23-7553 alone, however, did not produce cells with this apoptotic morphology.

ILX 23-7553 Interacts Additively with both Adriamycin and Irradiation.

ILX 23-7553 in combination with adriamycin or irradiation produces DNA fragmentation, apoptotic morphology, decreased cell viability and decreased clonogenic capacity. However, the shift in the dose response curves shown in FIGS. 2A-B may simply reflect additive interactions between ILX 23-7553 and adriamycin or irradiation. The inventors therefore determined whether the observed effects were occurring through a synergistic or additive interaction between ILX 23-7553 and adriamycin or irradiation. Statistical analysis of the results of two colony forming assays, one in which a single dose of ILX 23-7553 (200 nM) was combined with multiple doses of adriamycin (1-100 nM) and another in which a single dose of adriamycin (20 nM) was combined with varying doses ILX 23-7553 (0-200 nM). The results of these assays were compared to those predicted by the statistical model of additivity (Table 1A). The observed surviving fractions were determined to not be significantly different from those predicted by the model of additivity (p=0.461). Therefore, the assumption of additivity was not rejected and ILX 23-7553 and adriamycin were concluded to interact additively and not synergistically.

Statistical analysis was also performed to evaluate the ILX 23-7553 and irradiation combination. Results predicted by the model of additivity were compared to those observed using a single dose of ILX 23-7553 (200 nM) and various doses of irradiation 90.5-5 Gy) as well as a single dose of irradiation (2.5 Gy) and multiple doses of ILX 23-7553 (Table 1B). Similar to the adriamycin combination, the observed surviving fractions were not significantly different from those predicted using the model of additivity (p=0.196); therefore, the assumption of additivity was not rejected ILX 23-7553 and irradiation were concluded to interact additively.

TABLE 1A
ILX 23-7553 and Adriamycin Interact Additively

| Drug | Observed Mean | | |
|---|---|---|---|
| Combination | Response | Predicted Response | 95% Prediction |
| Adr (nM), | (Surviving | Under Additivity | Intervals Under |
| ILX (nM) | Fraction) | (Surviving Fraction) | Additivity |
| 20, 50 | 0.58 | 0.63 | [0.27, 0.99] |
| 20, 100 | 0.50 | 0.56 | [0.19, 0.93] |
| 1, 200 | 0.67 | 0.53 | [0.14, 0.93] |
| 5, 200 | 0.58 | 0.51 | [0.12, 0.91] |
| 10, 200 | 0.52 | 0.49 | [0.10, 0.88 |
| 20, 200 | 0.46 | 0.44 | [0.05, 0.83] |
| 100, 200 | 0.24 | 0.20 | [0, 0.60] |

Observed results from the colony forming assay were compared to values predicted using a statistical model of additivity. Observed and predicted values were not significantly different (mean p=0.46 1); the assumption of additivity is not rejected.

TABLE 1B
ILX 23-7553 and Irradiation Interact Additively

| Drug | Observed Mean | | |
|---|---|---|---|
| Combination | Response | Predicted Response | 95% Prediction |
| IR (Gy), | (Surviving | Under Additivity | Intervals Under |
| ILX (nM) | Fraction) | (Surviving Fraction) | Additivity |
| 2.5, 50 | 0.23 | 0.29 | [0, 0.38] |
| 0.5, 200 | 0.34 | 0.51 | [0, 0.61] |
| 1.0, 200 | 0.27 | 0.41 | [0.14, 0.88] |
| 2.5, 200 | 0.20 | 0.22 | [0.06, 0.77] |
| 5.0, 200 | 0.13 | 0.08 | [0, 0.55] |

Observed and predicted values were not significantly different (mean p=0.196); the assumption of additivity is not rejected.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference.

U.S. Pat. No. 6,087,350

U.S. Pat. No. 5,145,846

Bakhshi, Jensen, Goldman, Wright, McBride, Epstein, Korsmeyer, “Cloning the chromosomal breakpoint of t(14;18) human lymphomas: clustering around JH on chromosome 14 and near a transcriptional unit on 18,” Cell, 41(3):899-906, 1985.

Belleli et al., Carcinogenesis, 13, 2293 (1992).

Binderup et al., Biochem. Pharmacol., 42, 1569 (1991).

Binderup et al., page 192 in Proceedings of the 8th Workshop on Vitamin D, Paris France (Norman et al., Eds., Walter de Gruyter, Berlin) (1991).

Bower et al., Proc. Am. Assoc. Cancer. Res., 32, 1257 (1991).

Caldas, Hahn, da Costa, Redston, Schutte, Seymour, Weinstein, Hruban, Yeo, Kern, “Frequent somatic mutations and homozygous deletions of the p16 (MTS 1) gene in pancreatic adenocarcinoma,” Nat Genet, 8(1):27-32, 1994.

Calverley et al., “Vitamin D” in Antitumor Steroids (Blickenstaff, R., Ed., Academic Press, Orlando) (1992).

Cho et al., “Combined effects of 1,25 (OH)₂D₃ and platinum drugs on the growth of MCF-7 cells,” Cancer Res., 51:2848, 1991.

Clark et al., J. Cancer Res. Clin. Oncol., 118, 190 (1992).

Cleary and Sklar, “Nucleotide sequence of a t(14;18) chromosomal breakpoint in follicular lymphoma and demonstration of a breakpoint-cluster region near a transcriptionally active locus on chromosome 18,” Proc Natl Acad Sci USA, 82(21):7439-43, 1985.

Cleary, Smith, Sklar, “Cloning and structural analysis of cDNAs for bcl-2 and a hybrid bcl-2/immunoglobulin transcript resulting from the t(14; 18) translocation,” Cell, 47(1):19-28, 1986.

Colston et al., Lancet, 1, 188 (1989).

Cunningham et al., Br. J. Cancer, 63, 4673 (1991).

Elstner et al., Cancer Res., 55, 2822-30 (1995).

Fornari et al., “Growth arrest and non-apoptotic cell death associated with the suppression of c-myc expression in MCF-7 breast tumor cells following acute exposure to doxorubicin,” Biochem. Pharm., 51:931, 1996.

Fornari et al., “Induction of differentiation and growth arrest associated with nascent (nonoligosomal) DNA fragmentation and reduced c-myc expression in MCF-7 human breast tumor cells after continuous exposure to a sublethal concentration of doxorubicin,” Cell Growth Dif, 5:723, 1994.

Gavrieli, Sherman, Ben-Sasson, “Identification of programmed cell death in situ via specific labeling of nuclear DNA fraugmentation,” J. Cell Biol., 119:493, 1992.

Gennings et al., “Detection of departures from additivity in mixtures of many chemicals with a threshold model,” J. Agr. Bio. Env. State, 2:198, 1997.

Gewirtz et al., “Induction of DNA damage, inhibition of DNA synthesis and suppression of c-myc expression by the anthracycline analog, idarubicin (4-demethoxy-daunorubicin) in the MCF-7 breast tumor cell line,” Cancer Chemother. Pharmacol., 41:361, 1998.

Godyn et al., Cell Proliferation, 27, 37-46 (1994).

Hollstein, Sidransky, Vogelstein, Harris, “p53 mutations in human cancers,” Science, 253(5015):49-53, 1991.

Hussussian, Struewing, Goldstein, Higgins, Ally, Sheahan, Clark Jr, Tucker, Dracopoli, “Germline p16 mutations in familial melanoma,” Nat Genet, 8(1):15-21, 1994.

Kamb, Shattuck-Eidens, Eeles, Liu, Gruis, Ding, Hussey, Tran, Miki, Weaver-Feldhaus, “Analysis of the p16 gene (CDKN2) as a candidate for the chromosome 9p melanoma susceptibility locus,” Nat Genet, 8(1):23-6, 1994.

Kerr, Wyllie, Currie, “Apoptosis: a basic biological phenomenon with wide-ranging implications in tissue kinetics,” Br J Cancer, 26(4):239-57, 1972.

Mackie et al., Lancet, 342, 172, 1993.

McElwain et al., Mol. Cell. Diff., 3, 31-50, 1995.

Nobori, Miura, Wu, Lois, Takabayashi, Carson, “Deletions of the cyclin-dependent kinase-4 inhibitor gene in multiple human cancers,” Nature, 368(6473):753-6, 1994.

Okamoto, Demetrick, Spillare, Hagiwara, Hussain, Bennett, Forrester, Gerwin, Serrano, Beach, “Mutations and altered expression of p16INK4 in human cancer,” Proc Natl Acad Sci USA, (23):11045-91994, 1994.

Orlow, Zhou, Chakraborty, Drucker, Pifko-Hirst, Pawelek, “High-molecular-weight forms of tyrosinase and the tyrosinase-related proteins: evidence for a melanogenic complex,” J Invest Dermatol, 103(2): 196-201, 1994.

Radford et al., “Radiation response of mouse lymphoma and melanoma cells Part II. Apoptotic death is shown in all cell lines examined,” Int. J. Rad. Biol., 65:217, 1994.

Remington's Pharmaceutical Sciences, 18th Edition, Chapter 61, pages 1289-1329, 1990.

Rigby et al., J. Immunol., 135, 2279-86, 1985.

Saunders et al., Gynecol. Oncol., 51, 155-59, 1993.

Serrano et al., Nature, 366:704-707,1993.

Serrano et al., Science, 267:249-252,1995.

Sundaram and Gewirtz, “The vitamin D₃ analog EB 1089 enhances the response of human breast tumor cells to radiation,” Radiation Res., 152:479, 1999.

Sundaram et al., “EB 1089 enhances the antiproliferative and apoptotic effects of adriamycin in MCF-7 breast tumor cells,” Breast Cancer Res. And Treat., 63:1-10, 2000.

Tanaka et al., Clin. Orthopaed. Rel. Res., 247, 290-96, 1989.

Tsujimoto and Croce, “Analysis of the structure, transcripts, and protein products of bcl-2, the gene involved in human follicular lymphoma,” Proc Natl Acad Sci U S A. 83(14):5214-5218, 1986.

Tsujimoto, Cossman, Jaffe, Croce, “Involvement of the bcl-2 gene in human follicular lymphoma,” Science, 228(4706):1440-1443, 1985.

Wang et al., Cancer Res., 56, 264-67, 1996.

Watson et al., “The influence of ionizing radiation on proliferation, c-myc expression and the induction of apoptotic cell death in two breast tumor cell lines differing in p53 status,” Int. J. Rad. Biol., 72:547, 1997.

Weinberg, Science, 254:1138-1146, 1991.

Whitacre and Berger, “Factors affecting topotecan induced programmed cell death: adhesion protects cells from apoptosis and impairs cleavage of polyADP-ribose polymerase,” Cancer Res., 57:2157, 1997.

Wilson et al., “MCF-7 human mammary adenocarcinoma cell death in vitro in response to hormone withdrawal and DNA damage,” Int. J. Cancer, 51:502, 1995.

Wosikowski et al., “Normal p53 status and function despite the development of drug resistance in human breast cancer cells,” Cell Growth Diff., 6:139, 1995.

Zhou et al., Blood, 73, 75, 1991.

Zhou et al., Blood, 74, 82-93, 1989.

What is claimed is:

1. A method of inducing cell death in a cancer cell comprising administering to said cell:
a vitamin D₃ compound having the structure: 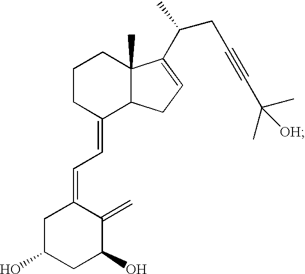
and ionizing radiation,
both in sufficient doses that, when combined, cell death is induced.

2. The method of claim 1, wherein said radiation is x-irradiation or γ-irradiation.

3. The method of claim 1, wherein said vitamin D₃ compound is provided prior to said radiation.

4. The method of claim 1, wherein said vitamin D₃ compound is provided after said radiation.

5. The method of claim 1, wherein said vitamin D₃ compound is provided concurrent with said radiation.

6. The method of claim 3, wherein said vitamin D₃ compound is administered less than one hour prior to said radiation.

7. The method of claim 3, wherein said vitamin D₃ compound is administered less than four hours prior to said radiation.

8. The method of claim 3, wherein said vitamin D₃ compound is administered less than eight hours prior to said radiation.

9. The method of claim 3, wherein said vitamin D₃ compound is administered less than 12 hours prior to said radiation.

10. The method of claim 3, wherein said vitamin D₃ compound is administered less than 24 hours prior to said radiation.

11. The method of claim 3, wherein said vitamin D₃ compound is administered less than 48 hours prior to said radiation.

12. The method of claim 4, wherein said vitamin D₃ compound is administered less than one hour after said radiation.

13. The method of claim 4, wherein said vitamin D₃ compound is administered less than four hours after said radiation.

14. The method of claim 4, wherein said vitamin D₃ compound is administered less than eight hours after said radiation.

15. The method of claim 4, wherein said vitamin D₃ compound is administered less than 12 hours after said radiation.

16. The method of claim 4, wherein said vitamin D₃ compound is administered less than 24 hours after said radiation.

17. The method of claim 4, wherein said vitamin D₃ compound is administered less than 48 hours after said radiation.

18. The method of claim 1, wherein said vitamin D₃ compound is formulated as about 0.5 to 1.0% of a pharmaceutical composition.

19. The method of claim 1, wherein said vitamin D₃ compound is formulated as about 0.8% of a pharmaceutical composition.

20. The method of claim 1, wherein said ionizing radiation is x-irradiation provided at fractionated doses of 2 Gy.

21. The method of claim 1, wherein said ionizing radiation is x-irradiation provided at a total dose of about 40 Gy.

22. The method of claim 1, wherein said ionizing radiation is x-irradiation provided at a total dose of about 60 Gy.

23. The method of claim 1, wherein said ionizing radiation is x-irradiation provided at a total dose of about 40 to 60 Gy.

24. The method of claim 1, wherein said cancer cell is a brain cancer cell, a liver cancer cell, a pancreatic cancer cell, a leukemia cell, a lymphatic cancer cell, a head & neck cancer cell, a lung cancer cell, a breast cancer cell, a thyroid cancer cell, a prostate cancer cell, a stomach cancer cell, a esophageal cancer cell, a colon cancer cell, a rectal cancer cell, a testicular cancer cell, a bladder cancer cell, a cervical cancer cell, an ovarian cancer cell or a skin cancer cell.

25. The method of claim 1, wherein the cancer cell is selected from the group consisting of an acute promyelocytic leukemia cell, a colon cancer cell, a rectal cancer cell and a liver cancer cell.

26. A method of treating a patient with cancer comprising administering to said patient:
a vitamin D₃ compound having the structure: 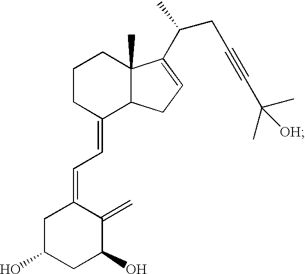
and ionizing radiation,
both in sufficient doses that, when combined, cancer cell death is induced, thereby treating cancer.

27. The method of claim 26, wherein said radiation is x-irradiation or γ-irradiation.

28. The method of claim 26, wherein said vitamin D₃ compound is provided prior to said radiation.

29. The method of claim 26, wherein said vitamin D₃ compound is provided after said radiation.

30. The method of claim 26, wherein said vitamin D₃ compound is provided concurrent with said radiation.

31. The method of claim 27, wherein said vitamin D₃ compound is administered about one hour prior to said radiation up to 48 hours prior to said radiation.

32. The method of claim 28, wherein said vitamin D₃ compound is administered less than one hour up to 48 hours after said radiation.

33. The method of claim 26, wherein said vitamin D₃ compound is provided at dose range of about 0.01 μg/kg body weight to about 2.5 μg/kg body weight.

34. The method of claim 26, wherein said vitamin D₃ compound is provided at dose range of about 0.1 μg/kg body weight to about 2.5 μg/kg body weight.

35. The method of claim 26, wherein said vitamin D₃ compound is provided at dose range of about 0.5 μg/kg body weight to about 2.5 μg/kg body weight.

36. The method of claim 26, wherein said vitamin D₃ compound is provided at dose range of about 1.0 μg/kg body weight to about 2.5 μg/kg body weight.

37. The method of claim 26, wherein said vitamin D₃ compound is provided at dose range of about 0.01 μg/kg body weight to about 1.0 μg/kg body weight.

38. The method of claim 26, wherein said ionizing radiation is x-irradiation provided at fractionated doses of 2 Gy.

39. The method of claim 26, wherein said ionizing radiation is x-irradiation provided at a total dose of about 40 to 60 Gy.

40. The method of claim 26, wherein said cancer is a brain cancer, a liver cancer, a pancreatic cancer, a leukemia, a lymphatic cancer, a head & neck cancer, a lung cancer, a breast cancer, a thyroid cancer, a prostate cancer, a stomach cancer, a esophageal cancer, a colon cancer, a rectal cancer, a testicular cancer, a bladder cancer, a cervical cancer, an ovarian cancer or a skin cancer.

41. The method of claim 26, wherein the cancer is selected from the group consisting of acute promyelocytic leukemia, colon cancer, rectal cancer and liver cancer.

42. The method of claim 26, wherein said patient is further treated with surgery, chemotherapy, immunotherapy, gene therapy or hormonal therapy.

43. The method of claim 26, wherein said patient has previously received a cancer therapy.

44. The method of claim 26, wherein said cancer is recurrent cancer.

45. A method of rendering a non-resectable tumor resectable comprising administering to a patient having said tumor:
a vitamin D₃ compound having the structure: 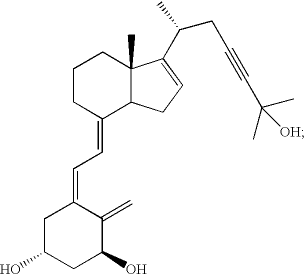
and ionizing radiation,
both in sufficient doses that, when combined, tumor cells are killed and said tumor is rendered resectable.

46. The method of claim 45, further comprising resecting said tumor.

47. A method of treating a patient with metastatic cancer comprising administering to said patient:
a vitamin D₃ compound having the structure: 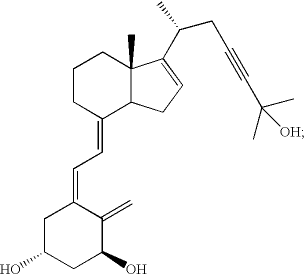
and ionizing radiation,
both in sufficient doses that, when combined, cancer cell death is induced, thereby reducing the number of metastases.

48. A method of reducing the tumor burden in a patient comprising administering to said patient:
a vitamin D₃ compound having the structure: 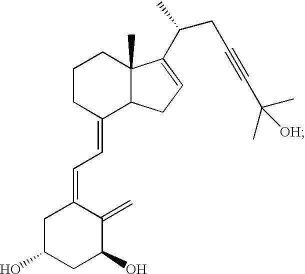
and ionizing radiation,
both in sufficient doses that, when combined, tumor cells are killed and said tumor burden is reduced.

49. A method of sensitizing a tumor cell to radiation comprising (a) selecting a tumor cell for radiation therapy and (b) administering to said tumor cell a vitamin D₃ compound having the structure:

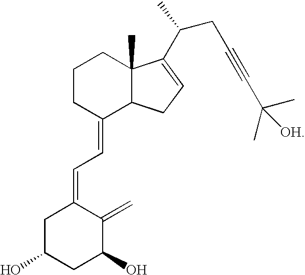

50. A method of reducing the radiation dose response curve for a tumor cell comprising (a) selecting a tumor cell for radiation therapy and (b) administering to said tumor cell a vitamin D₃ compound having the structure:

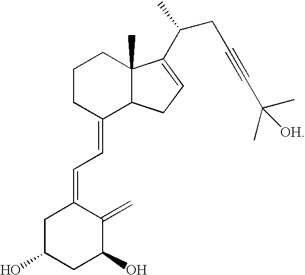

51. A method of overcoming radiation resistance in a tumor cell comprising (a) selecting a radiation resistant tumor cell for radiation therapy and (b) administering to said tumor cell a vitamin D₃ compound having the structure:

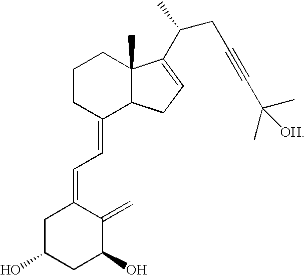

52. A method of inhibiting tumor angiogenesis in a patient comprising administering to said patient:
a vitamin D₃ compound having the structure: 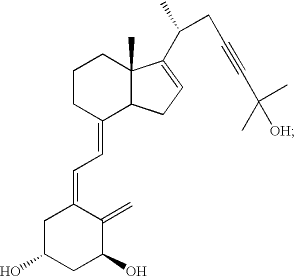
and ionizing radiation,
both in sufficient doses that, when combined, angiogenesis is inhibited.
